(19) United States
(12) Patent Application Publication
(10) Pub. No.: US 2023/0206013 A1
(43) Pub. Date: Jun. 29, 2023

(54) PATIENT-MONITORING DEVICES USING RFID TECHNOLOGY FOR USE IN PATIENT-MONITORING SYSTEMS

(71) Applicant: Murata Vios, Inc., Woodbury, MN (US)

(72) Inventor: Dattatraya BATHE, Woodbury, MN (US)

(21) Appl. No.: 18/089,161

(22) Filed: Dec. 27, 2022

Related U.S. Application Data
(60) Provisional application No. 63/293,970, filed on Dec. 27, 2021.

(51) Int. Cl. G06K 7/10; G06K 19/07; G16H 40/63

(52) U.S. Cl. CPC G06K 7/10366; G06K 19/0723; G16H 40/63

(57) ABSTRACT

A patient-monitoring device includes a sensor that includes a sensor identification and a removable pack that connects to and disconnects from the sensor. The removable pack includes a radio-frequency identification (RFID) tag that transmits a removable-pack identification and a memory that stores RFID data that is associated with the removable-pack identification. When the sensor is connected to a patient, the sensor senses patient data, and the patient-monitoring device communicates the patient data, the sensor identification, and the RFID data to a remote computer system.

### BACKGROUND OF THE INVENTION

### 1. Field of the Invention

The present invention relates to patient-monitoring devices that can sense patient data and that can communicate the sensed patient data to a remote computing system. More specifically, the present invention relates to patient-monitoring devices that use radio-frequency identification (RFID) technology to communicate with handheld mobile devices and that use a wireless local area network (WLAN), which can be implemented using, for example, wireless fidelity (WiFi) technology or other suitable radio communication technologies, to communicate with a remote computing system.

### 2. Description of the Related Art

FIG. 1 shows a known patient-monitoring system that includes a patient-monitoring device HW, a bedside monitor BSM, and a central server CS. The patient-monitoring device HW can be attached to a patient to sense and transmit patient data, including, for example, the patient’s physiological data. The patient-monitoring device HW and the beside monitor BSM can be included in a personal area network (PAN) that communicates using Bluetooth® technology. The beside monitor BSM can be a tablet, for example. The beside monitor BSM can be located in the patient’s room to provide access to and/or to display the patient data. A WLAN can include both the beside monitor BSM and the central server CS in which communications are performed electrically using WiFi technology. The known patient-monitoring system can also include a central station monitor CSM and/or a remote monitoring service RMS that are connected to the central server CS. The central station monitor CSM can be included in the WLAN and can communicate with the central server CS using WiFi technology. To display the correct patient data, a caregiver can use the beside monitor BSM to associate a patient-monitoring device HW with a patient ID of the patient so that the central server CS can send the correct patient data to the central station monitor CSM.

Problems with the known patient-monitoring system are that it requires a costly bedside monitor BSM for each patient, that it requires using Bluetooth® technology that requires pairing between devices, which can be slow and/or difficult to achieve pairing, it requires that the patient and the beside monitor BSM be within the small communication range of Bluetooth®, it requires that the beside monitor BSM be installed at the patient’s bedside, and that it does not allow for easy use of handheld mobile devices.

In a patient-monitoring system without a costly bedside monitor BSM, the caregiver still must communicate to the central server CS an association between the patient-monitoring device HW and the patient ID so that the central server CS can send the correct patient data.

A possible solution is to use a barcode or quick-response (QR) code on the patient-monitoring device HW, but a barcode or QR code requires all of an optical reader such as a camera or other similar optical device, a line-of-sight between the barcode and the optical reader, and sufficient light for the optical reader to read the barcode or QR code.

### SUMMARY OF THE INVENTION

To overcome the problems described above, preferred embodiments of the present invention provide patient-monitoring devices that each can be used in patient-monitoring systems and methods to send sensed patient data to a remote computing system and that can use RFID technology to communicate with a handheld mobile device so that the sensed patient data can be displayed on the handheld mobile device. By using RFID technology, patient-monitoring devices according to preferred embodiments of the present invention avoid the problems with using Bluetooth® technology, barcodes, and QR codes discussed above.

According to a preferred embodiment of the present invention, a patient-monitoring device includes a sensor that includes a sensor identification and a removable pack that connects to and disconnects from the sensor. The removable pack includes a radio-frequency identification (RFID) tag that transmits a removable-pack identification and a memory that stores RFID data that is associated with the removable-pack identification. When the sensor is connected to a patient, the sensor senses patient data, and the patient-monitoring device communicates the patient data, the sensor identification, and the RFID data to a remote computer system.

When the removable pack is connected to the sensor, the sensor can receive the RFID data from the removable pack and can communicate the RFID data to the remote computer system. The removable pack can include a battery and/or a metal plate that covers or substantially covers the sensor. The memory can be a read-only memory. The RFID data can include the removable-pack identification. The RFID tag can be readable by a handheld mobile device so that, when the removable-pack identification is received by the handheld mobile device, the patient data can be displayed on the handheld mobile device.

According to a preferred embodiment of the present invention, a patient-monitoring system includes the patient-monitoring device of one of the various preferred embodiments of the present invention, the remote computing system, and a handheld mobile device that reads the RFID tag to receive the removable-pack identification and that transmits the removable-pack identification to the remote computing system. The remote computing system is programmed and/or configured to receive the sensor identification after the sensor is attached to the patient; to receive the RFID data from the patient-monitoring device after the removable pack is connected to the sensor; to receive the removable-pack identification from the handheld mobile device; to associate the sensor identification with a patient identification of the patient for a first period during which the patient-monitoring device is connected to the patient; based on the RFID data, to associate the removable-pack identification with the patient identification for a second period during which the removable pack is connected to the sensor; and after receiving the removable-pack identification from the handheld mobile device, to send patient data to the handheld mobile device.

The first period can be longer than the second period. The first period can be more than approximately 24 hours, and the second period can be less than approximately 24 hours. The patient-monitoring system can further include a wireless local area network that can include the sensor, the handheld mobile device, and the remote computing system. The patient-monitoring system can further include a remote monitoring system connected to the remote computing system.

According to a preferred embodiment of the present invention, a patient-monitoring method includes attaching a patient-monitoring device to a patient for a first period, the patient-monitoring device includes a sensor that includes a sensor identification and that senses patient data; attaching a first removable pack to the sensor for a second period less than the first period, where the first removable pack includes a first radio-frequency identification (RFID) tag that transmits a first-removable-pack identification and a first memory that stores first RFID data that is associated with the first-removable-pack identification; after the second period, removing the first removable pack and attaching a second removable pack to the sensor, where the second removable pack includes a second radio-frequency identification (RFID) tag that transmits a second-removable-pack identification and a second memory that stores second RFID data that is associated with the second-removable-pack identification; and electrically communicating the patient data, the sensor identification, the first RFID data, and the second RFID data to a remote computing system to: during the first period, associate the sensor identification with a patient identification of the patient; during the second period and based on the first RFID data, associate the first removable-pack identification with the patient identification; and after the second period and based on the second RFID data, changing an association of the patient identification from the first-removable-pack identification to the second-removable-pack ID.

The patient-monitoring method can further include using a handheld mobile device to, during the second period, read the first RFID tag to receive the first-removable-pack identification and transmit the first removable-pack identification to the remote computing system; and after the second period, read the second RFID tag to receive the second-removable-pack identification and transmit the second removable-pack identification to the remote computing system; and using the remote computer system to, during the second period and after receiving the first-removable-pack identification, send patient data to the handheld mobile device; and after the second period and after receiving the second-removable-pack identification, send patient data to the handheld mobile device.

The removable pack can include a battery and/or metal plate that covers or substantially covers the sensor. The first and the second memories can be read-only memories. The first RFID data can include the first-removable-pack identification, and the second RFID data can include the second-removable-pack identification. The first period can be more than approximately 24 hours, and the second period can be less than approximately 24 hours.

The patient-monitoring method can further included transmitting and receiving data using a wireless local area network that includes the sensor, the handheld mobile device, and the remote computing system. The patient-monitoring method can further include transmitting and receiving data using a remote monitoring system connected to the remote computing system.

The above and other features, elements, characteristics, steps, and advantages of the present invention will become more apparent from the following detailed description of preferred embodiments of the present invention with reference to the attached drawings.

### BRIEF DESCRIPTION OF THE DRAWINGS

FIG. 1 is a diagram of a known patient monitoring system including sensor hardware that communicates via Bluetooth to a bedside monitor.

FIG. 2 is a diagram of a patient-monitoring system including a sensor device that can communicate using RFID technology and using a WLAN.

FIGS. 3 and 4 are front and back views of a sensor device that can be used in the patient-monitoring system of FIG. 2.

FIG. 5 is a perspective view of a removable pack used in the sensor device of FIG. 3.

FIG. 6 is a perspective view of the removable pack of FIG. 5 without a top housing.

FIG. 7 is a front perspective view of the top housing of the removable pack of FIG. 5.

FIG. 8 is a back perspective view of the top housing of FIG. 7 with an RFID tag.

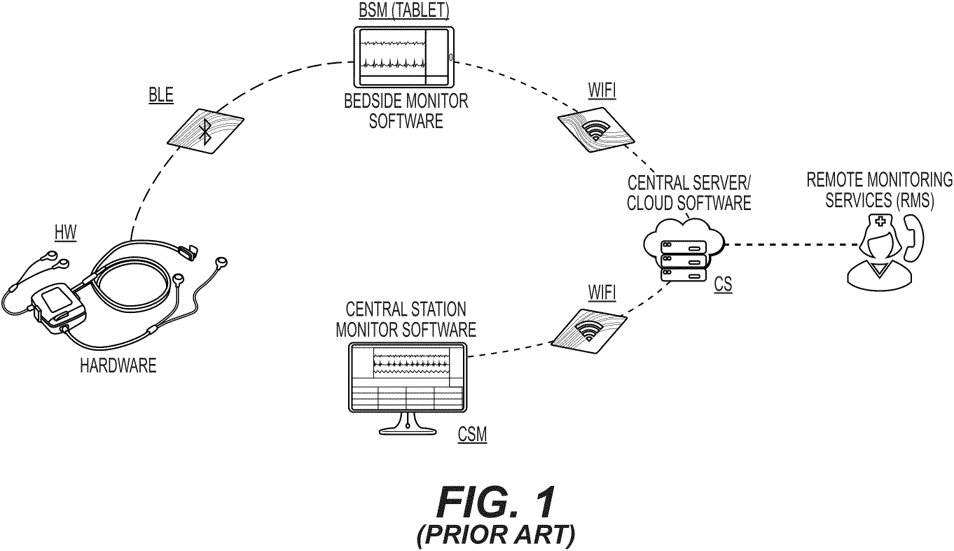

### DETAILED DESCRIPTION OF PREFERRED EMBODIMENTS

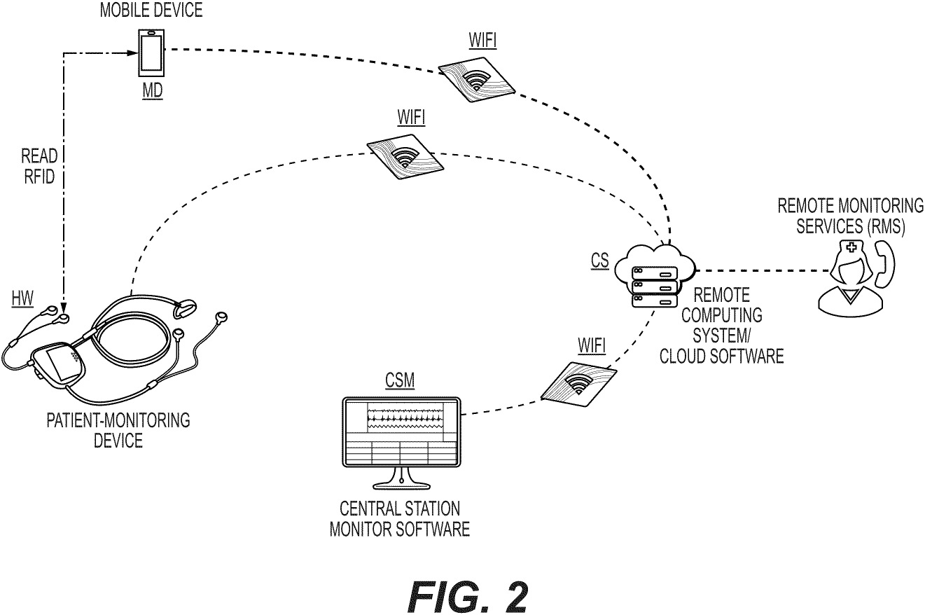

FIG. 2 shows a patient-monitoring system that includes a patient-monitoring device HW, a handheld mobile device MD, and a remote computing system CS. The patient-monitoring device HW includes a sensor that is attached to the patient, and while the sensor is attached to a patient, different removable packs can be connected to and disconnected from the sensor. A caregiver can use a handheld device to read RFID tags connected to the different removable packs. When the caregiver uses a handheld mobile device MD to read the RFID tag on the removable pack connected to the sensor, the remote computing system CS should send the correct patient data to the caregiver’s handheld mobile device MD. To be able to send the correct patient data to the caregiver’s handheld mobile device MD, the remote computing system CS needs to dynamically associate (1) the removable-pack ID that uniquely identifies the removable pack connected to the sensor with (2) the patient ID that uniquely identifies the patient. Sending the correct patient data can be accomplished if the patient-monitoring device HW sends RFID data stored in the new removable pack that uniquely identifies the new removable pack after the new removable pack is connected to the sensor but before the caregiver uses the handheld mobile device MD to read the RFID tag one or in the new removable pack.

The patient-monitoring system of FIG. 2 can include a WLAN that includes the patient-monitoring device HW, the handheld mobile device MD, and the remote computing system CS. The WLAN can use WiFi technology or other similar radio communication technology to communicate. The patient-monitoring system of FIG. 2 can also include a central station monitor CSM and/or a remote monitoring service RMS that are connected to the central server CS. The central station monitor CSM can be included in the WLAN and can communicate with the central server CS using WiFi technology or other similar technology.

The handheld mobile device MD can include, for example, a mobile telephone or other similar device that can read RFID tags and that can display data.

The remote computing system CS can be any suitable computing system or systems that can be programmed and/or configured to provide the functions described below. The remote computing CS can be located in the facility in which the patient is located or can be cloud-based servers. The remote computing system CS can be located “remote” from the patient. For example, the remote computing system CS can be located in the same facility, e.g., the same hospital, as the patient, but not in the same room as the patient. The remote computing system CS can also include an access point that is connected to cloud-based servers so that the patient data is sent from the patient-monitoring device HW to the cloud-based servers and is sent from the cloud-based servers to the handheld mobile device MD.

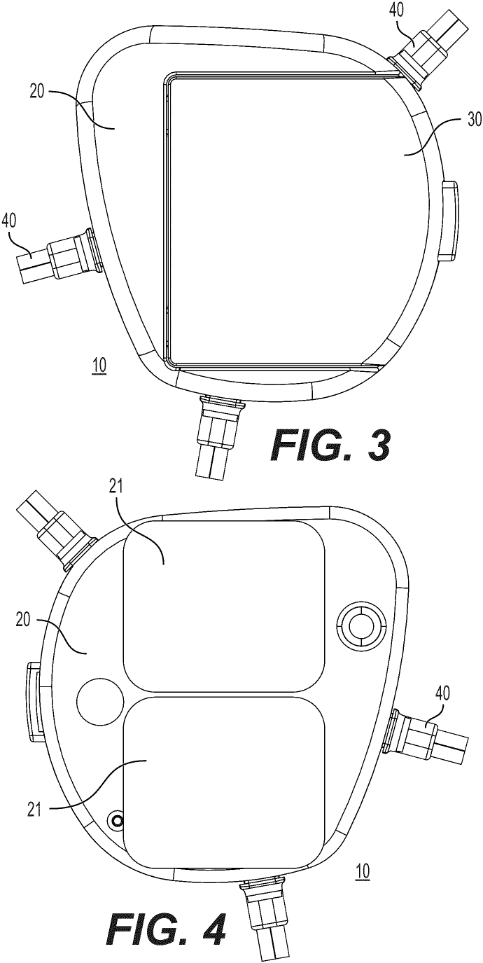

A patient-monitoring device 10 is shown in FIGS. 3 and 4. The patient-monitoring device 10 can communicate with the remote computing system CS using, for example, WiFi technology of a WLAN. The patient-monitoring device 10 can include a sensor 20 that can be connected to and disconnected from a patient and can include a removable pack 30 that can be connected to and disconnected from the sensor 20. The sensor 20 and/or the removable pack 30 can include a communication module that allows data to be sent and/or received. The communication module can, for example, use WiFi technology. Any suitable sensor 20 can be used, including, for example a chest sensor that can sense data related to a patient’s heart and/or breathing.

The sensor 20 can include leads 40 that are connected to electrodes that can be attached to a patient’s skin. FIGS. 3 and 4 show three leads 40, but any number of leads 40 can be used. As shown in FIG. 4, the sensor 20 can also include electrodes 21 on the back of the sensor 20 that can be attached to a patient’s skin. FIG. 4 shows two electrodes 21, but any number of electrodes 21 can be used. The sensor 20 includes a memory that stores a sensor ID that uniquely identifies the sensor 20. In the remote computing system CS, the sensor ID can be associated with a patient ID that uniquely identifies the patient to which the patient-monitoring device 10 is attached. For example, a caregiver can manually associate the sensor ID with the patient ID in the remote computing system CS or can use a handheld mobile device to associate the sensor ID with the patient ID. If the old sensor 20 is disconnected from the patient and a new sensor 20 is connected to the patient, then a new association between a new sensor ID of the new sensor 20 and the patient ID can be created, and the old sensor ID can be no longer associated with the patient ID. The new association between the new sensor ID and the patient ID can be created manually or through the handheld mobile device.

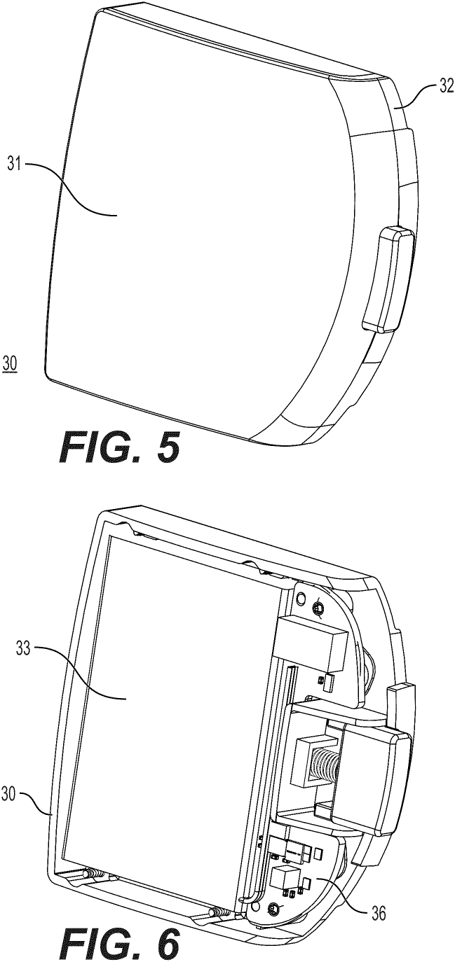

The removable pack 30 is shown in FIGS. 5 and 6. The removable pack 30 can include a battery 33 but is not required to do so. It is preferable to keep the sensor 20 connected to the patient’s skin and to avoid having to re-attach the sensor 20 to reduce the chance of irritating the patient’s skin. If the removable pack 30 includes a battery 33, then a new removable pack 30 can be connected to the sensor 20 after a certain amount of time has passed or when the battery 33 is discharged to a predetermined level. If a battery 33 is used, then any suitable battery 33 can be used, including, for example, a rechargeable battery. Changing the battery 33 powering the sensor 20 allows the sensor 20 to stay connected to the patient’s skin so that the patient data can be continuously sensed. As a non-limiting example, the sensor 20 can be connected to a patient for a first time period of more than approximately 24 hours, while the removable pack 30 can be connected to the sensor 20 for a second time period of less than approximately 24 hours, allowing the battery 33 powering the sensor 20 to be replaced approximately every 24 hours.

As shown in FIG. 5, the removable pack 30 can include top housing 31 and bottom housing 32. The top housing 31 and bottom housing 32 can be made of plastic or any other suitable material. FIG. 6 shows the removable pack 30 without the top housing 31. In FIG. 6, the removable pack 30 includes a battery 33 and a printed circuit board (PCB) 36. A memory can be connected to the PCB 36 for storing data, including, as explained below, data that can be used to uniquely identify the removable pack 30. Any suitable memory can be used, including, for example, a read-only memory such as electrically erasable programmable read-only memory (EEPROM).

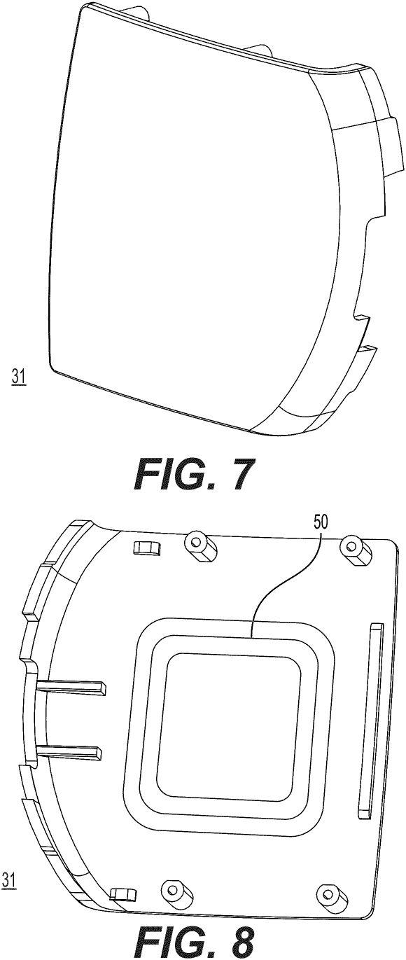

The top housing 31 is shown in FIGS. 7 and 8. As shown in FIG. 8, an RFID tag 50 can be applied to an interior surface of the top housing 31. The RFID tag 50 can be used to transmit a removable-pack ID that uniquely identifies the removable pack 30. The handheld mobile device can read the RFID tag 50 on or in the removable pack 30 to receive the removable-pack ID and can transmit removable-pack ID to the remote computing system CS.

Using an RFID tag 50 as opposed to using a barcode or QR code allows the removable pack 30 to be identified easily by bringing the handheld mobile device into close proximity to the RFID tag 50 without having to use an optical reader, ensuring line-of-sight between the optical reader and the barcode or QR code, or ensuring adequate light to read the barcode or QR code.

The memory connected to the PCB 36 in the removable pack 30 can store the RFID data that is associated with removable-pack ID so that the removable pack 30 can be uniquely identified. Typically, the RFID data will be the same as the removable-pack ID, but the RFID data does not have to be the same as the removable-pack ID so long as the RFID data can be used to identify the associated removable pack 30. The RFID data can be stored in the memory during the manufacturing of the removable pack 30. When the removable pack 30 is connected to the sensor 20, the RFID data can be transmitted from the memory to the sensor 20, and the sensor 20 can transmit the sensor ID, the RFID data, and the patient data to the remote computing system CS. It is also possible that, if the communication module is included in the removable pack 30, the removable pack 30 receives the patient data and sensor ID from the sensor 20, and the removable pack 30 can transmit the sensor ID, the RFID data, and the patient data to the remote computing system CS. Data can be transmitted between the sensor 20 and the removable pack 30 using any suitable communication protocol, including, for example, inter-integrated circuit (I2C) protocol.

The removable pack 30 can cover or substantially cover the sensor 20 as shown in FIG. 3, where substantially cover can mean, for example, that the removable pack 30 covers at least approximately 70% of the sensor 20. If the removable pack 30 includes a battery 33 or a metal plate, then an RFID tag 50 cannot be placed on the sensor 20 because the battery 33 or the metal plate would interfere with reading the RFID tag 50.

For simplicity, an example will be explained in which a patient-monitoring device 10 is connected to a patient for a first period and a first removable pack 30 is connected to the sensor 20 for a second period that is less than the first period, which requires that a second removable pack 30 be connected to the sensor 20 after the second period.

First, the patient-monitoring device 10 can be connected to the patient, and the patient-monitoring device 10 can electrically communicate the sensor ID to the remote computing system. The first removable pack 30 can be connected to the sensor 20 before the patient-monitoring device 10 is connected to the patient or can be connected after the sensor 20 is connected to the patient. The patient-monitoring device 10 can electrically communicate the first RFID data to the remote computing system.

The remote computing system can then associate the sensor ID and the first RFID data with the patient ID.

During the second time period, when a caregiver reads the RFID tag 50 of the first removable pack 30 with a handheld mobile device, the handheld mobile device sends the first removable-pack ID to the remote computing system. Because the first-removable-pack ID is the same as or is associated with the first RFID data, the remote computing system can send the correct patient data to the handheld mobile device so that the handheld mobile device can display the correct patient data to the caregiver.

After the second period, the first removable pack 30 can be removed from the sensor 20, and the second removable pack 30 can be connected to the sensor 20. The patient-monitoring device 10 can then electrically communicate the second RFID data to the remote computing system. The remote computing system can dynamically change the associations of the patient ID, removing the association with the first RFID data and adding an association with the second RFID data.

After the second time period, when a caregiver reads the RFID tag 50 of the second removable pack 30 with a handheld mobile device, the handheld mobile device can send the second-removable-pack ID to the remote computing system. Because the second-removable-pack ID is the same as or is associated with the second RFID data, the remote computing system can send the correct patient data to the handheld mobile device so that the handheld mobile device can display the correct patient data to the caregiver.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1. A patient-monitoring device comprising:
a sensor that includes a sensor identification; and
a removable pack that connects to and disconnects from the sensor, the removable pack includes:
a radio-frequency identification (RFID) tag that transmits a removable-pack identification; and
a memory that stores RFID data that is associated with the removable-pack identification, wherein
when the sensor is connected to a patient:
the sensor senses patient data; and
the patient-monitoring device communicates the patient data, the sensor identification, and the RFID data to a remote computer system.

2. The patient-monitoring device of claim 1, wherein, when the removable pack is connected to the sensor, the sensor receives the RFID data from the removable pack and communicates the RFID data to the remote computer system.

3. The patient-monitoring device of claim 1, wherein the removable pack includes a battery and/or a metal plate that covers or substantially covers the sensor.

4. The patient-monitoring device of claim 1, wherein the memory is a read-only memory.

5. The patient-monitoring device of claim 1, wherein the RFID data includes the removable-pack identification.

6. The patient-monitoring device of claim 1, wherein the RFID tag is readable by a handheld mobile device so that, when the removable-pack identification is received by the handheld mobile device, the patient data is displayed on the handheld mobile device.

7. A patient-monitoring system comprising:
the patient-monitoring device of claim 1;
the remote computing system; and
a handheld mobile device that reads the RFID tag to receive the removable-pack identification and that transmits the removable-pack identification to the remote computing system, wherein
the remote computing system is programmed and/or configured to:
receive the sensor identification after the sensor is attached to the patient;
receive the RFID data from the patient-monitoring device after the removable pack is connected to the sensor;
receive the removable-pack identification from the handheld mobile device;
associate the sensor identification with a patient identification of the patient for a first period during which the patient-monitoring device is connected to the patient;
based on the RFID data, associate the removable-pack identification with the patient identification for a second period during which the removable pack is connected to the sensor; and
after receiving the removable-pack identification from the handheld mobile device, send patient data to the handheld mobile device.

8. The patient-monitoring system of claim 7, wherein the first period is longer than the second period.

9. The patient-monitoring system of claim 8, wherein the first period is more than approximately 24 hours, and the second period is less than approximately 24 hours.

10. The patient-monitoring system of claim 7, further comprising a wireless local area network that includes the sensor, the handheld mobile device, and the remote computing system.

11. The patient-monitoring system of claim 7, further comprising a remote monitoring system connected to the remote computing system.

12. A patient-monitoring method comprising:
attaching a patient-monitoring device to a patient for a first period, the patient-monitoring device includes a sensor that includes a sensor identification and that senses patient data;
attaching a first removable pack to the sensor for a second period less than the first period, the first removable pack includes:
a first radio-frequency identification (RFID) tag that transmits a first-removable-pack identification; and
a first memory that stores first RFID data that is associated with the first-removable-pack identification;
after the second period, removing the first removable pack and attaching a second removable pack to the sensor, the second removable pack includes:
a second radio-frequency identification (RFID) tag that transmits a second-removable-pack identification; and
a second memory that stores second RFID data that is associated with the second-removable-pack identification; and
electrically communicating the patient data, the sensor identification, the first RFID data, and the second RFID data to a remote computing system to:
during the first period, associate the sensor identification with a patient identification of the patient;
during the second period and based on the first RFID data, associate the first removable-pack identification with the patient identification; and
after the second period and based on the second RFID data, changing an association of the patient identification from the first-removable-pack identification to the second-removable-pack ID.

13. The patient-monitoring method of claim 12, further comprising:
using a handheld mobile device to:
during the second period, read the first RFID tag to receive the first-removable-pack identification and transmit the first removable-pack identification to the remote computing system; and
after the second period, read the second RFID tag to receive the second-removable-pack identification and transmit the second removable-pack identification to the remote computing system; and
using the remote computer system to:
during the second period and after receiving the first-removable-pack identification, send patient data to the handheld mobile device; and
after the second period and after receiving the second-removable-pack identification, send patient data to the handheld mobile device.

14. The patient-monitoring method of claim 12, wherein the removable pack includes a battery and/or metal plate that covers or substantially covers the sensor.

15. The patient-monitoring method of claim 12, wherein the first and the second memories are read-only memories.

16. The patient-monitoring method of claim 12, wherein
the first RFID data includes the first-removable-pack identification, and
the second RFID data includes the second-removable-pack identification.

17. The patient-monitoring method of claim 12, wherein the first period is more than approximately 24 hours, and the second period is less than approximately 24 hours.

18. The patient-monitoring method of claim 12, further comprising transmitting and receiving data using a wireless local area network that includes the sensor, the handheld mobile device, and the remote computing system.

19. The patient-monitoring method of claim 12, further comprising transmitting and receiving data using a remote monitoring system connected to the remote computing system.
